(19) United States
(12) Patent Application Publication
(10) Pub. No.: US 2026/0253715 A1
(43) Pub. Date: Aug. 27, 2026

(54) LEVERAGING LARGE LANGUAGE MODELS FOR STANDARDIZING CLINICAL DATA

(71) Applicants: Genentech, Inc., South San Francisco, CA (US); Hoffmann-la Roche Inc., Little Falls, NJ (US)

(72) Inventors: Yogesh P. Pandit, Fremont, CA (US); Arindam Sett, San Leandro, CA (US); Mrunal Shriram Yadav, Milton (CA); Somaye Sadat Hashemifar, Sunnyvale, CA (US); Seyed Mohammadmoshen Hejrati, Stanford, CA (US)

(21) Appl. No.: 19/682,725

(22) Filed: May 20, 2026

Related U.S. Application Data
(63) Continuation of application No. PCT/US2024/057117, filed on Nov. 22, 2024.
(60) Provisional application No. 63/605,066, filed on Dec. 1, 2023.

(51) Int. Cl. G16H 40/20; G06F 16/334; G16H 10/60

(52) U.S. Cl. CPC G16H 40/20; G06F 16/3347; G16H 10/60

(57) ABSTRACT

Disclosed are various embodiments for leveraging large language models (LLMs) and retrieval-augmented generation (RAG) for data standardization of clinical AI data. A query comprising a target dataset that needs to be standardized and a dataset dictionary can be obtained. Augmented data can be extracted from an external source. A prompt including the query and the augmented data can be generated and applied to a large language model configured to output a response to the query. The response can include the target dataset standardized according to a standard format.

### CROSS-REFERENCE TO RELATED APPLICATIONS

This application is a continuation application of PCT Application No. PCT/US2024/057117, filed on Nov. 22, 2024, which application claims priority to, and the benefit of, U.S. Provisional Patent Application No. 63/605,066 filed on 1 Dec. 2023.

### BACKGROUND

The development of Artificial Intelligence (AI) in healthcare has the potential to improve clinical outcomes, reduce costs, and enhance patient satisfaction. However, a major challenge associated with the use of AI in healthcare is the standardization of clinical data. In particular, using clinical datasets from disparate sources with different schemas (e.g., data structures) and data dictionaries (e.g., information about data types and their meanings) can pose several challenges in AI development including data inconsistencies, feature mismatches, data quality issues, information losses, processing complexities, legal and ethical issues, interoperability issues, etc.

With respect to data inconsistences, different sources may have different conventions for documenting the same information. This inconsistency can lead to data mishandling, which in turn jeopardizes the accuracy of AI models. Feature mismatches can pose a challenge if different datasets use different features, or represent identical features differently. For example, it can be difficult to reconcile them into a uniform model. Even similar types of data can be represented differently, making harmonization challenging. Data quality issues can be a concern when datasets from different sources often have different levels of quality and reliability. Some datasets may contain more errors or inaccuracies than others, which can affect the performance of the AI models. Information Loss can further cause challenges. In particular, during the process of standardizing or transforming the data to fit into a single schema, some important data can be lost. Another challenge in AI development is that managing data from disparate sources increases the overall complexity of data processing. This can further lead to increased time and resources spent on data wrangling. The use of disparate data source can also raise concerns about privacy and data security. Furthermore, the algorithms developed using the data can be unknowingly biased due to the variations in the quality of data from these sources. To minimize potential bias and maximize the effectiveness of LLMs for various applications, prompt engineering is an emerging field that focuses on creating and fine-tuning prompts for LLMs. It involves a comprehensive set of techniques that boost interaction with LLMs, enhance their safety, integrate domain knowledge, and work with external tools. However, interoperability issues can also pose challenges. For example, different healthcare information systems tend to use different data standards, creating compatibility issues and limiting how freely data can be exchanged between systems. Ultimately, such challenges may compromise the validity, generalizability, and reliability of AI solutions in the clinical field. This emphasizes the importance of a robust, harmonized data sourcing and integration strategy in clinical AI development.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing depicting one of several embodiments of the present disclosure.

FIG. 2 is a drawing of a network environment according to various embodiments of the present disclosure.

FIG. 3 is a flowchart illustrating one example of functionality implemented as portions of an application executed in a computing environment in the network environment of FIG. 2 according to various embodiments of the present disclosure.

### DETAILED DESCRIPTION

Disclosed are various approaches for using large language models (LLMs) for data standardization of clinical AI data. In various examples, LLMs can be used to identify and map medical concept entities to existing data standards such as, for example, Fast Healthcare Interoperability Resources (FHIR). Leveraging the LLMs for data standardization can significantly reduce the burden of manual data mapping and increase the efficiency and accuracy of the data standardization process. According to various examples, the present disclosure relates to leveraging large language models for mapping any raw datasets to a clinical data standard (e.g., FHIR). In various examples, a zero/few-shot learning approach is leveraged to achieve data mapping for standardization. The systems and methods of the present disclosure can accelerate the adoption of AI in healthcare and improve the quality of care for patients.

The adoption of data standards for clinical data has become increasingly important with the rise of artificial intelligence (AI) applications in healthcare. To develop accurate and effective AI models for tasks like diagnosis, prognosis, and treatment recommendations, high-quality structured clinical data is required. However, clinical data has historically been challenging to standardize due to its complexity and heterogeneity.

Previous research has extensively explored the use of AI in clinical data standardization, primarily focusing on rule based methodologies and conventional machine learning techniques. These approaches have paved the way for notable advancements in handling structured data. However, they often fall short when dealing with complicated domain-specific schemas, which constitute a significant portion of real-world clinical datasets. Techniques like natural language processing have been applied to extract information from unstructured text or data cleansing. The potential of these LLMs for semantic understanding and context-aware data mapping suggests they could offer substantial improvements in the field of clinical data standardization.

In recent years, Fast Healthcare Interoperability Resources (FHIR) has emerged as a leading standard for clinical data exchange. FHIR provides a common framework and set of APIs for representing and sharing clinical data in a standardized way. FHIR acts as a bridge for AI, allowing it to extract structured clinical data from electronic health records and other healthcare systems in a consistent format. Some key benefits of FHIR include (1) structured data format based on resources with common fields for clinical concepts like patients, conditions, medications, etc., enabling integration and analysis across datasets; (2) integration and analysis enabled across datasets; (3) modular components that can be used in flexible ways to represent various clinical workflows, facilitating interoperability across systems; (4) interoperability facilitated across systems; (5) modern web standards and APIs that can be used for efficient data access and exchange; and (6) an active open source community with rapid evolution of specifications.

For AI specifically, FHIR provides the ability to extract structured clinical variables from electronic health records and other systems in a standardized way. This helps address the “data wrangling” challenges that often dominate healthcare AI projects. With more adoption of FHIR, higher-quality datasets will become available for developing, evaluating, and deploying AI algorithms. In summary, data standards like FHIR are foundational for realizing the potential of AI in healthcare. By enabling interoperability and data accessibility, FHIR can help guide the responsible and effective application of AI to drive better clinical outcomes and patient care. Ongoing adoption of FHIR will be crucial for advancing clinical AI innovation in the years ahead.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principals disclosed by the following illustrative examples.

According to various examples, the system of the present disclosure uses an LLM with Retrieval-Augmented Generation (RAG) to provide a common framework for defining a consistent data terminology across various datasets and institutions. This standardization is essential for interoperability, enabling healthcare organizations to exchange patient data seamlessly, researchers to aggregate and analyze data consistently, and developers to create applications that work with healthcare information reliably.

In various examples, the system of the present disclosure takes the dictionary of a target dataset as an input and generates an output that conforms to a standard format (e.g., FHIR), resulting in a standardized format for the target dataset. In various examples, the data dictionary of the target dataset can include definitions, descriptions, explanations of terms, fields, variables used in the dataset, and/or other data. In various examples, RAG uses a semantic search to retrieve relevant context (e.g., augmented data) that LLMs use to produce more accurate responses.

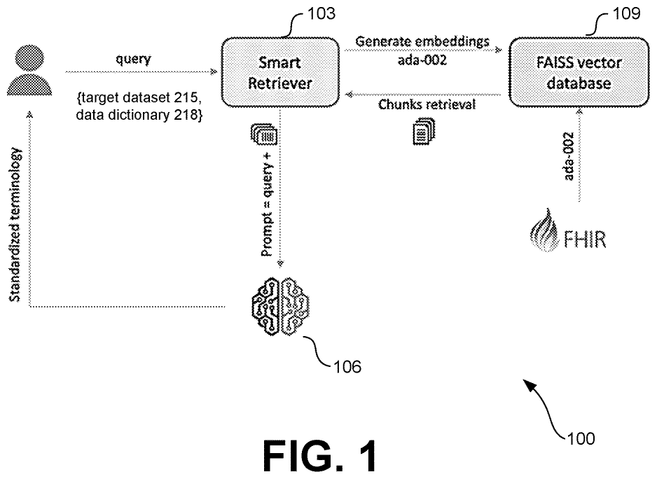

Turning now to FIG. 1, shown is an example schematic of a system 100 for data standardization. As shown in FIG. 1, the system 100 can comprise a retriever service 103 and a LLM 106. In various examples, the retriever service 103 can obtain a query from a user associated with a computing device. The query can include the target dataset 215 that is to be conformed to the standard format and a data dictionary 218 associated with the target dataset 215. The retriever service 103 can further extract relevant document snippets (e.g., augmented data) from a database 109 of documents of the desired format (e.g., FHIR document database). The LLM 106 can represent any language model that includes a neural network with many parameters (tens of thousands, millions, or sometimes even billions or more) that is trained on large quantities of unlabeled text using self-supervised learning or semi-supervised learning techniques. Examples of large language models 106 include various versions of OPENAI's Generative Pre-trained Transformer (GPT) model (e.g., GPT-1, GPT-2, GPT-3, GPT-4, etc.), META's Large Language Model Meta AI (LLaMA), and GOOGLE's Pathways Language Model 2 (PaLM 2), among others. In various examples, the LLM 106 can be configured to produce answers (e.g., format the target dataset 215) using the information from the snippets retrieved from the database 109 of documents. In essence, RAG helps the LLM 106 by searching and retrieving contextual information associated with the standard format (e.g., FHIR) to improve its (i.e., LLM's) responses.

In various examples, the formatted documents (e.g., the FHIR documents) can be split into smaller chunks using various splitting algorithms (e.g., recursive character text splitting, character-based splitting, token-based splitting, semantic splitting, etc.). The chunks can then be embedded in a vector space. For example, the chunks can be embedded using the OpenAI text-embedding-ada-002 embedding engine and/or other type of embedding engine and stored in a vector database 109 (e.g., the Facebook AI Similarity Search (FAISS 1.7.4) vector database). In another example, a resource-element pair can be embedded into a vector database using chunking. In various examples, the vector database 109 is used to find the k-most similar chunks to a given query at the query time. The original query (e.g., target dataset 215 and data dictionary 218), combined with the retrieved chunks (e.g., augmented data) can be compiled into a prompt and passed to the LLM 106 for generating the answer. This provides the LLM with additional information that contains factual data, which can help to improve the quality of its responses. Accordingly, the output of the LLM 106 can include the data in the target dataset 215 that is conformed to the standard format.

In various examples, the system 100 of the present disclosure predicts the structures for the data dictionary 218 of a dataset 215. Each structure consists of several metadata blocks, with the first block usually being the resource. The system 100 and corresponding LLM 106 were tested based on the number of matched metadata blocks between the predicted structure and the actual (i.e., groundtruth) structure. The performance for top-k predictions was tested where k=3, 4, 5.

During testing, a predicted structure is categorized as an ‘Absolute Match’ if all of its blocks align with those in the actual structure (e.g., the groundtruth). If the resource aligns but some or all other metadata elements do not, this leads to a ‘Partial Match’. Alternatively, if none of the blocks match, or if the resource does not match even when other elements do, the predicted structure is considered a ‘Mismatch’. Partial Score and Match Score are then defined as below:

$$PartialScore = \frac{S + P}{N} \times 100$$

$$P = {\sum }_{i = 1}^{K} \frac{intersecton ( {pred}_{i} , {gt}_{i} )}{union ( {pred}_{i} , g {t}_{i} )}$$

Where S and N represent the quantity of absolute matches and the total structures in the dataset dictionary, respectively. P is the fraction of matched blocks in all ‘Partial Match’ structures. predᵢ and gtᵢ correspond to the predicted and groundtruth values for structure i, respectively. K is the number of all ‘Partial Match’ structures. A Resource Match Score is also calculated based on instances where the resources matched between groundtruth and Predicted results. Similar to Partial Score, the Resource Match Score is also a percentage value.

$$Resource Match Score = \frac{S + K}{N} \times 100$$

Where S, K, and N represent the quantity of absolute matches, the quantity of ‘Partial Match’ structures, and the total structures in the dataset dictionary, respectively. Tables 1 and 2 represent the results of the evaluation with and without RAG.

TABLE 1
Performance of System With RAG

| | Absolute | Partial | | Partial | Match |
|---|---|---|---|---|---|
| Dataset | Match | Match | Mismatch | Score | Score |
| ADNI | 74 | 126 | 58 | 52.97 | 77.52 |

TABLE 2
Performance of System Without RAG

| | Absolute | Partial | | Partial | Match |
|---|---|---|---|---|---|
| Dataset | Match | Match | Mismatch | Score | Score |
| ADNI | 75 | 121 | 62 | 51.97 | 75.97 |

Table 3 presents the results for top-k=20, averaged over 10 mapping iterations to account for the variations in results in individual mapping run. The model exhibited strong overall performance with a mean score of 73.54 (SD=0.16) and a low standard deviation of 0.11 for the resource match score (94.52). This consistency indicates that the model performs reliably across various datasets and exhibits minimal variability between runs. The resource match score, which evaluates the alignment between predicted resources and the groundtruth, shows variability across datasets. While some datasets demonstrate high resource match scores (e.g., ADNI_DOD with 97.20), others exhibit lower alignment (e.g., CERA with 89.8). The consistency in resource match scores, as indicated by small standard deviations, indicates that the model maintains a relatively stable performance in resource prediction across different datasets. Some datasets exhibit notably high scores, such as ADNI_DOD (77.93), COVASTIL (78.05), BIOFINDER1 (77.09), and BLAZE (79.59), while others, like ADNI (51.31), LUNG_PET_CT_DX (52.86) display lower performance.

The standard deviations accompanying these scores provide insight into the consistency of the model's performance, indicating relatively small variations across individual runs. While the model maintains consistency, there remains variability in its efficacy across different datasets, as evident in the significant deviation in scores, especially for datasets like LUNG_PET_CT_DX, NLST, LIDC-IDRI, and CERA. In one aspect, the quality of the data dictionary and the specific FHIR profiles used could influence the variability in efficacy. An informative and comprehensive dictionary could equip the LLM to interpret even cryptic column names effectively, facilitating better mapping and mitigating performance variability.

TABLE 3
Scores for Datasets Mapped to FHIR (top-k
= 20) averaged over 10 mapping iterations

| | | | Resource Match | |
|---|---|---|---|---|
| Dataset | Score(%) | | Score(%) | |
| A4 | 57.16 | (±0.77) | 94.36 | (±0.34) |
| ADNI | 51.31 | (±1.01) | 66.58 | (±1.24) |
| ADNI_DOD | 77.93 | (±0.2) | 97.20 | (±0.18) |
| AIBL | 73.26 | (±2.15) | 90.89 | (±1.86) |
| BIOFINDER1 | 77.09 | (±0.53) | 95.84 | (±0.45) |
| BLAZE | 79.59 | (±0.94) | 96.70 | (±0.55) |
| CERA | 73.17 | (±3.05) | 89.8 | (±2.75) |
| CITELINE | 64.18 | (±2.9) | 82.39 | (±2.69) |
| COVASTIL | 78.05 | (±0.36) | 96.03 | (±0.38) |
| HABS | 71.75 | (±0.49) | 95.15 | (±0.4) |
| LIDC-IDRI | 72.77 | (±3.6) | 84.00 | (±3.77) |
| NLST | 67.48 | (±2.59) | 84.6 | (±2.13) |
| TAURIEL | 71.20 | (±0.47) | 94.09 | (±0.28) |
| LUNG_PET_CT_DX | 52.86 | (±4.23) | 68.00 | (±3.77) |
| Total | 73.54 | (±0.16) | 94.52 | (±0.11) |

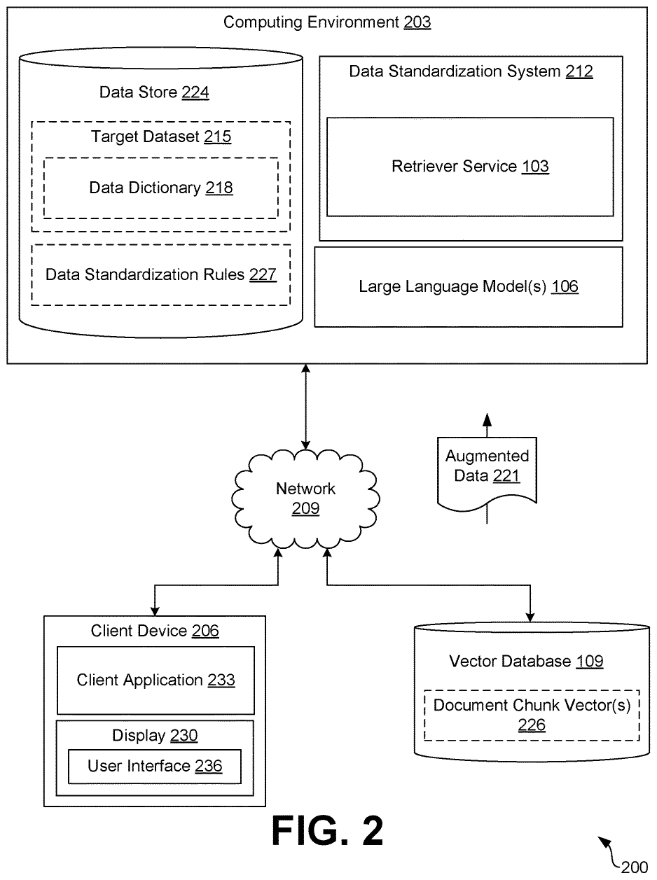

With reference to FIG. 2, shown is a network environment 200 according to various embodiments. The network environment 200 can include a computing environment 203, a vector database 109, and a client device 206, which can be in data communication with each other via a network 209.

The network 209 can include wide area networks (WANs), local area networks (LANs), personal area networks (PANs), or a combination thereof. These networks can include wired or wireless components or a combination thereof. Wired networks can include Ethernet networks, cable networks, fiber optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (i.e., WI-FI®), BLUETOOTH® networks, microwave transmission networks, as well as other networks relying on radio broadcasts. The network 209 can also include a combination of two or more networks 209. Examples of networks 209 can include the Internet, intranets, extranets, virtual private networks (VPNs), and similar networks.

The computing environment 203 can include one or more computing devices that include a processor, a memory, and/or a network interface. For example, the computing devices can be configured to perform computations on behalf of other computing devices or applications. As another example, such computing devices can host and/or provide content to other computing devices in response to requests for content.

Moreover, the computing environment 203 can employ a plurality of computing devices that can be arranged in one or more server banks or computer banks or other arrangements. Such computing devices can be located in a single installation or can be distributed among many different geographical locations. For example, the computing environment 203 can include a plurality of computing devices that together can include a hosted computing resource, a grid computing resource or any other distributed computing arrangement. In some cases, the computing environment 203 can correspond to an elastic computing resource where the allotted capacity of processing, network, storage, or other computing-related resources can vary over time.

Various applications or other functionality can be executed in the computing environment 203. The components executed on the computing environment 203 include the data standardization system 212, one or more large language models (LLMs) 106, and other applications, services, processes, systems, engines, or functionality not discussed in detail herein.

The data standardization system 212 can be executed to obtain data to be translated to a standard format. For example, the data standardization system 212 can obtain a query from a user or entity associated with a client device 206. The query can include the target dataset 215 that is to be conformed to the standard format and a data dictionary 218 associated with the target dataset 215.

The data standardization system 212, via the retriever service 103, can further be executed to obtain augmented data 221 from the vector database 109. For example, the retriever service 102 can extract relevant document snippets (e.g., augmented data 221) from the vector database 109 that comprises a database of documents of the desired format (e.g., FHIR document database). In various examples, the retriever service 103 can generate embeddings based at least in part on the data dictionary 218 and/or the target dataset 215. The embeddings can be used to compare with vector embeddings stored in the vector database 109 to identify document snippets that are formatted in the standard format. The document snippets obtained from the vector database 109 can be used as augmented data 221 for standardizing the target dataset 215.

In various examples, the data standardization system 212 can generate a prompt that is input to an LLM based at least in part on the data dictionary 218, the target dataset 215, and the augmented data 221. In various examples, the data standardization system 212 can execute the LLM 106 or otherwise apply the generated prompt as an input to the LLM 106 to generate an answer to the original query (e.g., standardize the target dataset 215 into the standard format).

A large language model (LLM) 106 can represent any language model that includes a neural network with many parameters (tens of thousands, millions, or sometimes even billions or more) that is trained on large quantities of unlabeled text using self-supervised learning or semi-supervised learning techniques. Some large language models 106 may be generative—that is they can generate new data based at least in part on patterns and structure learned from their input training data. Examples of large language models 106 include various versions of OPENAI's Generative Pre-trained Transformer (GPT) model (e.g., GPT-1, GPT-2, GPT-3, GPT-4, etc.), META's Large Language Model Meta AI (LLAMA), and GOOGLE's Pathways Language Model 2 (PaLM 2), among others. A large language model 106 can be configured to return a response to a prompt, which can be in a structured form (e.g., a request or prompt with a predefined schema and/or parameters) or in an unstructured form (e.g., free form or unstructured text).

Also, various data is stored in a data store 224 that is accessible to the computing environment 203. The data store 224 can be representative of a plurality of data stores 224 which can include relational databases or non-relational databases such as object-oriented databases, hierarchical databases, hash tables or similar key-value data stores, as well as other data storage applications or data structures. Moreover, combinations of these databases, data storage applications, and/or data structures may be used together to provide a single, logical, data store. The data stored in the data store 224 is associated with the operation of the various applications or functional entities described below. This data can include the target dataset 215, data standardization rules 227, and potentially other data.

The data standardization rules 227 can include rules, models, and/or configuration data for the various algorithms or approaches employed by the data standardization system 212. For example, the data standardization rules 227 can be employed by the data standardization system in defining the standardized format for the target dataset 215. The data standardization rules 227 can further be employed by the data standardization system in defining how the embeddings for obtaining the document chunks (e.g., augmented data 221) from the vector database 109.

The vector database 109 can comprise a database storing document chunk vectors 226 that represent text chunks of documents in the standard format. In various examples, the formatted documents (e.g., the FHIR documents) can be split into text chunks using recursive character text splitting or other text splitting algorithms. The chunks can then be embedded in a vector space. For example, the chunks can be embedded using the OpenAI text-embedding-ada-002 embedding engine. The vector database 109 can comprise a vector database such as, for example, the Facebook AI Similarity Search (FAISS 1.7.4) vector database and/or other type of vector database. In various examples, the vector database 109 is used to find the k-most similar chunks to a given query at the query time.

The client device 206 is representative of a plurality of client devices that can be coupled to the network 209. The client device 206 can include a processor-based system such as a computer system. Such a computer system can be embodied in the form of a personal computer (e.g., a desktop computer, a laptop computer, or similar device), a mobile computing device (e.g., personal digital assistants, cellular telephones, smartphones, web pads, tablet computer systems, music players, portable game consoles, electronic book readers, and similar devices), media playback devices (e.g., media streaming devices, BluRay® players, digital video disc (DVD) players, set-top boxes, and similar devices), a videogame console, or other devices with like capability. The client device 206 can include one or more displays 230, such as liquid crystal displays (LCDs), gas plasma-based flat panel displays, organic light emitting diode (OLED) displays, electrophoretic ink (“E-ink”) displays, projectors, or other types of display devices. In some instances, the display 230 can be a component of the client device 206 or can be connected to the client device 206 through a wired or wireless connection.

The client device 206 can be configured to execute various applications such as a client application 233 or other applications. The client application 233 can be executed in a client device 206 to access network content served up by the computing environment 203 or other servers, thereby rendering a user interface 236 on the display 230. To this end, the client application 233 can include a browser, a dedicated application, or other executable, and the user interface 236 can include a network page, an application screen, or other user mechanism for obtaining user input. The client device 206 can be configured to execute applications beyond the client application 233 such as email applications, social networking applications, word processors, spreadsheets, or other applications.

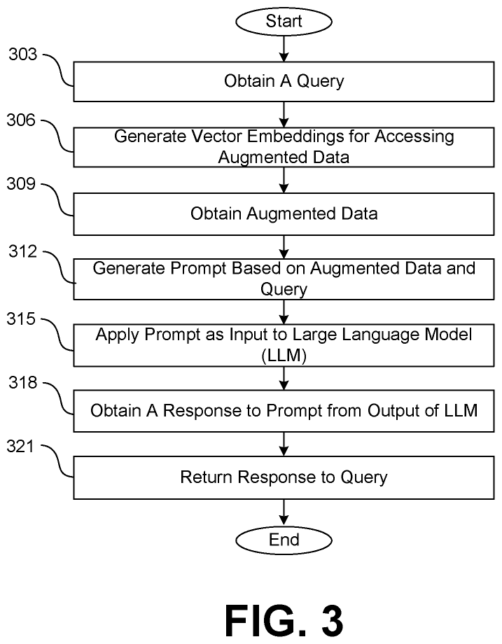

Next, a general description of the operation of the various components of the network environment 200 is provided with respect to FIG. 3. FIG. 3 is a flowchart that provides one example of the operation of a portion of the data standardization system 212. The flowchart of FIG. 3 provides merely an example of the many different types of functional arrangements that can be employed to implement the operation of the depicted portion of the data standardization system 212. As an alternative, the flowchart of FIG. 3 can be viewed as depicting an example of elements of a method implemented within the network environment 200.

Beginning with block 303, the data standardization system 212 obtains a query. In various examples, the query comprise a target dataset 215 and a corresponding data dictionary 218. In various examples, the target dataset 215 can include clinical AI data and the data dictionary 218 can include definitions, descriptions, explanations of terms, fields, variables used in the dataset, and/or other data. In some examples, the query is obtained from a data store 224. In other examples, the query is obtained from a client device 206. For example, a user interacting with a user interface 236 of the client device 206 can generate the query by defining the target dataset 215 and corresponding data dictionary 218 that needs to conform with a standardized format. Accordingly, the query can be transmitted by the client device 206 to the data standardization system 212.

At block 306, the data standardization system 212 can generate vector embeddings for accessing augmented data 221 from the vector database 109. In various examples, the data standardization system 212 uses retrieval-augmented generation (RAG) with an LLM 106 for determining the answer (e.g., standardized data) to the query. Accordingly, the data standardization system 212 can generate vector embeddings associated with the target dataset 215 and the data dictionary 218 that are associated with the type of data that needs to be formatted or otherwise standardized according to the standardized format.

At block 309, the data standardization system 212 can obtain the augmented data 221 from the vector database 109 based at least in part on the vector embeddings. For example, the vector embeddings associated with the target dataset 215 can be compared to stored vector embeddings in the vector database 109 to identify the appropriate document chunk vectors 226 that should be included in the augmented data 221. In particular, the data standardization system 212 uses Retriever Service 103 to further extract relevant document snippets (e.g., augmented data 221) from the vector database 109 of documents of the desired format (e.g., FHIR document database).

At block 312, the data standardization system 212 can generate a prompt based at least in part on the augmented data 221 and the query. For example, the original query (e.g., target dataset 215 and data dictionary 218), combined with the retrieved chunks (e.g., augmented data 221) can be compiled into a prompt that can be applied as an input to a LLM 106. In essence, RAG (e.g., the use of the augmented data 221) can help the LLM 106 to search and retrieve contextual information associated with the standard format (e.g., FHIR) to improve its responses.

At block 315, the data standardization system 212 applies the prompt as an input to the LLM 106. The LLM 106 can be trained to standardize the format of the target dataset 215 into a desired format. In essence, RAG (e.g., the use of the augmented data 221) can help the LLM 106 to search and retrieve contextual information associated with the standard format (e.g., FHIR) to improve its responses (e.g., standardizing the format of the target dataset 215).

At block 318, the data standardization system 212 can obtain the response to the prompt from the output of the LLM 106. In various examples, the response can comprise the target dataset 215 that has been standardized into a given format.

At block 321, the response to the query can be returned. For example, if the query is obtained from a client device 206, the response can be transmitted to the client device 206. In other examples, the response can be stored in a data store 224. Accordingly, the reformatted or otherwise standardized dataset can be used as subsequent training data, therefore improving the performance of AI models that analyze the standardized dataset. Thereafter, this portion of the process proceeds to completion.

A number of software components previously discussed are stored in the memory of the respective computing devices and are executable by the processor of the respective computing devices. In this respect, the term “executable” means a program file that is in a form that can ultimately be run by the processor. Examples of executable programs can be a compiled program that can be translated into machine code in a format that can be loaded into a random-access portion of the memory and run by the processor, source code that can be expressed in proper format such as object code that is capable of being loaded into a random-access portion of the memory and executed by the processor, or source code that can be interpreted by another executable program to generate instructions in a random-access portion of the memory to be executed by the processor. An executable program can be stored in any portion or component of the memory, including random-access memory (RAM), read-only memory (ROM), hard drive, solid-state drive, Universal Serial Bus (USB) flash drive, memory card, optical disc such as compact disc (CD) or digital versatile disc (DVD), floppy disk, magnetic tape, or other memory components.

The memory includes both volatile and nonvolatile memory and data storage components. Volatile components are those that do not retain data values upon loss of power. Nonvolatile components are those that retain data upon a loss of power. Thus, the memory can include random-access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, or other memory components, or a combination of any two or more of these memory components. In addition, the RAM can include static random-access memory (SRAM), dynamic random-access memory (DRAM), or magnetic random-access memory (MRAM) and other such devices. The ROM can include a programmable read-only memory (PROM), an erasable programmable read-only memory (EPROM), an electrically erasable programmable read-only memory (EEPROM), or other like memory device.

Although the applications and systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart shows the functionality and operation of an implementation of portions of the various embodiments of the present disclosure. If embodied in software, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as a processor in a computer system. The machine code can be converted from the source code through various processes. For example, the machine code can be generated from the source code with a compiler prior to execution of the corresponding application. As another example, the machine code can be generated from the source code concurrently with execution with an interpreter. Other approaches can also be used. If embodied in hardware, each block can represent a circuit or a number of interconnected circuits to implement the specified logical function or functions.

Although the flowchart shows a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowchart can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that includes software or code can be embodied in any non-transitory computer-readable medium for use by or in connection with an instruction execution system such as a processor in a computer system or other system. In this sense, the logic can include statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. Moreover, a collection of distributed computer-readable media located across a plurality of computing devices (e.g, storage area networks or distributed or clustered filesystems or databases) may also be collectively considered as a single non-transitory computer-readable medium.

The computer-readable medium can include any one of many physical media such as magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium can be a random-access memory (RAM) including static random-access memory (SRAM) and dynamic random-access memory (DRAM), or magnetic random-access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device, or in multiple computing devices in the same computing environment 203.

In addition to the foregoing, the various embodiments of the present disclosure include, but are not limited to, the embodiments set forth in the following clauses.
Clause 1. A system, comprising a computing device comprising a processor and a memory; and machine-readable instructions stored in the memory that, when executed by the processor, cause the computing device to at least: obtain a query comprising a target dataset and a dataset dictionary; generate a plurality of vector embeddings based at least in part on the dataset dictionary and the target dataset; obtain augmented data from an external source based at least in part on the plurality of vector embeddings; generate a prompt comprising the query and the augmented data; apply the prompt to a large language model configured to output a response to the query based at least in part on the prompt; and transmit the response to the query to a computing device.
Clause 2. The system of clause 1, wherein the target dataset comprises clinical data.
Clause 3. The system of clause 1 or clause 2, wherein the external source comprises a vector database comprising a plurality of documents formatted according to a standardized format.
Clause 4. The system of clause 3, wherein the response comprises the target dataset formatted according to the standardized format.
Clause 5. The system of clause 4, wherein the standardized format comprises Fast Healthcare Interoperability Resources (FHIR) format.
Clause 6. The system of any one of clauses 1 to 5, wherein the prompt is generated based at least in part on Retrieval-Augmented Generation (RAG).
Clause 7. A method, comprising: obtaining, via at least one computing device, a query comprising a target dataset and a dataset dictionary; generating, via the at least one computing device, a plurality of vector embeddings based at least in part on the dataset dictionary and the target dataset; obtaining, via the at least one computing device, augmented data from an external source based at least in part on the plurality of vector embeddings; generating, via the at least one computing device, a prompt comprising the query and the augmented data; applying, via the at least one computing device, the prompt to a large language model configured to output a response to the query based at least in part on the prompt; and transmitting, via the at least one computing device, the response to the query to a computing device.
Clause 8. The method of clause 7, wherein the target dataset comprises clinical data.
Clause 9. The method of clause 7 or clause 8, wherein the external source comprises a vector database comprising a plurality of documents formatted according to a standardized format.
Clause 10. The method of clause 9, wherein the response comprises the target dataset formatted according to the standardized format.
Clause 11. The method of clause 10, wherein the standardized format comprises Fast Healthcare Interoperability Resources (FHIR) format.
Clause 12. The method of any one of clauses 7 to 11, wherein the prompt is generated based at least in part on Retrieval-Augmented Generation (RAG).
Clause 13. A non-transitory, computer-readable medium, comprising machine-readable instructions that, when executed by a processor of a computing device, cause the computing device to at least: obtain a query comprising a target dataset and a dataset dictionary; generate a plurality of vector embeddings based at least in part on the dataset dictionary and the target dataset; obtain augmented data from an external source based at least in part on the plurality of vector embeddings; generate a prompt comprising the query and the augmented data; apply the prompt to a large language model configured to output a response to the query based at least in part on the prompt; and transmit the response to the query to a computing device.
Clause 14. The non-transitory, computer-readable medium of clause 13, wherein the target dataset comprises clinical data.
Clause 15. The non-transitory, computer-readable medium of clause 13 or clause 14, wherein the external source comprises a vector database comprising a plurality of documents formatted according to a standardized format.
Clause 16. The non-transitory, computer-readable medium of clause 15, wherein the response comprises the target dataset formatted according to the standardized format.
Clause 17. The non-transitory, computer-readable medium of any one of clauses 13 to 16, wherein the standardized format comprises Fast Healthcare Interoperability Resources (FHIR) format.
Clause 18. The non-transitory, computer-readable medium of any one of clauses 13 to 17, wherein the prompt is generated based at least in part on Retrieval-Augmented Generation (RAG).

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A system, comprising:
a computing device comprising a processor and a memory; and
machine-readable instructions stored in the memory that, when executed by the processor, cause the computing device to at least:
obtain a query comprising a target dataset and a dataset dictionary;
generate a plurality of vector embeddings based at least in part on the dataset dictionary and the target dataset;
obtain augmented data from an external source based at least in part on the plurality of vector embeddings;
generate a prompt comprising the query and the augmented data;
apply the prompt to a large language model configured to output a response to the query based at least in part on the prompt; and
transmit the response to the query to a computing device.

2. The system of claim 1, wherein the target dataset comprises clinical data.

3. The system of claim 1, wherein the external source comprises a vector database comprising a plurality of documents formatted according to a standardized format.

4. The system of claim 3, wherein the response comprises the target dataset formatted according to the standardized format.

5. The system of claim 4, wherein the standardized format comprises Fast Healthcare Interoperability Resources (FHIR) format.

6. The system of claim 1, wherein the prompt is generated based at least in part on Retrieval-Augmented Generation (RAG).

7. A method, comprising:
obtaining, via at least one computing device, a query comprising a target dataset and a dataset dictionary;
generating, via the at least one computing device, a plurality of vector embeddings based at least in part on the dataset dictionary and the target dataset;
obtaining, via the at least one computing device, augmented data from an external source based at least in part on the plurality of vector embeddings;
generating, via the at least one computing device, a prompt comprising the query and the augmented data;
applying, via the at least one computing device, the prompt to a large language model configured to output a response to the query based at least in part on the prompt; and
transmitting, via the at least one computing device, the response to the query to a computing device.

8. The method of claim 7, wherein the target dataset comprises clinical data.

9. The method of claim 7, wherein the external source comprises a vector database comprising a plurality of documents formatted according to a standardized format.

10. The method of claim 9, wherein the response comprises the target dataset formatted according to the standardized format.

11. The method of claim 10, wherein the standardized format comprises Fast Healthcare Interoperability Resources (FHIR) format.

12. The method of claim 7, wherein the prompt is generated based at least in part on Retrieval-Augmented Generation (RAG).

13. A non-transitory, computer-readable medium, comprising machine-readable instructions that, when executed by a processor of a computing device, cause the computing device to at least:
obtain a query comprising a target dataset and a dataset dictionary;
generate a plurality of vector embeddings based at least in part on the dataset dictionary and the target dataset;
obtain augmented data from an external source based at least in part on the plurality of vector embeddings;
generate a prompt comprising the query and the augmented data;
apply the prompt to a large language model configured to output a response to the query based at least in part on the prompt; and
transmit the response to the query to a computing device.

14. The non-transitory, computer-readable medium of claim 13, wherein the target dataset comprises clinical data.

15. The non-transitory, computer-readable medium of claim 13, wherein the external source comprises a vector database comprising a plurality of documents formatted according to a standardized format.

16. The non-transitory, computer-readable medium of claim 15, wherein the response comprises the target dataset formatted according to the standardized format.

17. The non-transitory, computer-readable medium of claim 13, wherein the standardized format comprises Fast Healthcare Interoperability Resources (FHIR) format.

18. The non-transitory, computer-readable medium of claim 13, wherein the prompt is generated based at least in part on Retrieval-Augmented Generation (RAG).
